(19) United States
(12) Patent Application Publication
(10) Pub. No.: US 2014/0366887 A1
(43) Pub. Date: Dec. 18, 2014

(54) ORAL AIRWAY

(71) Applicant: Tufts Medical Center, Inc., Boston, MA (US)

(72) Inventor: Pei-Shan Zhao, Needham, MA (US)

(73) Assignee: Tufts Medical Center, Inc., Boston, MA (US)

(21) Appl. No.: 14/241,918

(22) PCT Filed: Aug. 30, 2012
(86) PCT No.: PCT/US12/53133

§ 371 Date: Jun. 13, 2014

Related U.S. Application Data
(60) Provisional application No. 61/528,902, filed on Aug. 30, 2011.

(51) Int. Cl. A61M 16/04

(52) U.S. Cl. CPC A61M 16/0465; A61M 16/0495
USPC 128/207.14

(57) ABSTRACT

An oral airway includes a curved body having a front wall which includes a concave portion of the body, and a rear wall which includes a convex portion of the body. The front wall is configured to translate relative to the rear wall, while remaining engaged with the rear wall. In addition, the rear wall is formed of a first material, and at least a portion of the front wall is formed of a second material, the second material being relatively flexible as compared to the first material.

### BACKGROUND OF THE INVENTION

Tracheal intubation refers to the placement of a flexible tube (e.g., an endotracheal tube) into the trachea of the body to protect the patient's airway and provide for mechanical ventilation. Fiberoptic intubation is one type of tracheal intubation, and involves threading an endotracheal tube over the shaft of a flexible fiberoptic scope. The fiberoptic scope is passed through the mouth of the patient, into the pharynx, and through the vocal chords into the patient's trachea. Upon visual confirmation of tracheal rings and carina, the fiberoptic scope is held steady while the endotracheal tube is advanced over the fiberoptic scope into the patient's airway. Once the endotracheal tube is in place, the fiberoptic scope is removed, and the patient is ventilated.

The base of the tongue frequently blocks the view of the larynx and vocal chords during fiberoptic intubation. In addition, difficult intubation is often encountered when the patient's larynx is more anterior than normal. Fiberoptic intubation is still the standard approach used in these situations. However, some oral airway devices currently used for fiberoptic intubations may not be optimal because they are formed as a rigid, fixed tube, and can not push the base of the tongue away. To overcome this problem, anesthesia providers have to either manually lift the jaw, or more often, pull the patient's tongue out using their hand which may cause patient discomfort upon awakening.

### SUMMARY

In some aspects, an oral airway is provided that includes a curved body having a front wall which includes a concave portion of the body, and a rear wall which includes a convex portion of the body. The front wall is configured to translate relative to the rear wall, while remaining engaged with the rear wall, and the rear wall is formed of a first material, and at least a portion of the front wall is formed of a second material, the second material being relatively flexible as compared to the first material.

The oral airway may include one or more of the following features: The body further includes a proximal end, and a distal end opposed to the proximal end, and the front wall translates relative to the rear wall in a direction extending from the proximal end to the distal end. The front wall is separable from the rear wall. The body further includes a linear portion and a curved portion extending from an end of the linear portion, and at least a portion the front wall linear portion is formed of the second material. The front wall curved portion is formed of the first material. The front wall proximal end is formed of the second material. The front wall and rear wall are maintained in an assembled configuration by an interlocking structure. The interlocking structure comprises a series of spaced protrusions formed on a side edge of the rear wall, and mating protrusions formed on a corresponding side edge of the front wall, wherein the mating protrusions are configured to engage corresponding ones of the rear wall spaced protrusions. The body is tubular. The front wall corresponds to about one-third of the circumference of the oral airway, and the rear wall corresponds to about two-thirds of the circumference of the oral airway. An end of the linear portion includes an outwardly-protruding flange. The body further includes a proximal end, and a distal end opposed to the proximal end, a linear portion including the proximal end, and a curved portion extending between the linear portion and the distal end. In addition, the front wall is selectively movable between a retracted position and an advanced position, such that when in the retracted position, the front wall linear portion length is greater than the rear wall linear portion length, and the front wall curved portion length is less than the rear wall curved portion length, and when in the advanced position, the front wall linear portion length is substantially equal to the rear wall linear portion length, and the front wall curved portion length is greater than the rear wall curved portion length, where the wall length is the distance along the body between the proximal and distal ends. The oral airway is generally oval in cross-sectional shape.

In some aspects, an oral airway is provided that includes a curved body having a front wall which includes a concave portion of the body, a rear wall which includes a convex portion of the body, a proximal end, a distal end opposed to the proximal end, a linear portion including the proximal end, and a curved portion extending between the linear portion and the distal end. The front wall is configured to selectively translate relative to the rear wall, while remaining engaged with the rear wall, between a retracted position and an advanced position, such that when in the retracted position, the front wall linear portion length is greater than the rear wall linear portion length, and the front wall curved portion length is less than the rear wall curved portion length, and when in the advanced position, the front wall linear portion length is substantially equal to the rear wall linear portion length, and the front wall curved portion length is greater than the rear wall curved portion length.

The oral airway may include one or more of the following features: The front wall translates relative to the rear wall in a direction extending from the proximal end to the distal end. The front wall is separable from he rear wall. The front wall and rear wall are maintained in an assembled configuration by an interlocking structure. The interlocking structure comprises a series of spaced protrusions formed on a side edge of the rear wall, and mating protrusions formed on a corresponding side edge of the front wall, wherein the mating protrusions are configured to engage corresponding ones of the rear wall spaced protrusions. The body is tubular. The rear wall is formed of a first material, and at least a portion of the front wall is formed of a second material, the second material being relatively flexible as compared to the first material. At least a portion the front wall linear portion is formed of the second material. The front wall curved portion is formed of the first material. The front wall proximal end is formed of the second material. The front wall distal end is formed of the first material. The oral airway is generally oval in cross-sectional shape.

Among other advantages, because the front wall of the oral airway translates relative to the airway body, the distal end of the front wall can serve as a laryngoscopy blade to push the base of the tongue up, and thereby improve the ability to visualize the larynx and vocal chords.

The airway body is a generally rigid structure including a linear portion and curved portion. Advantageously, at least a portion of the front wall of the body is formed of a material that is relatively flexible as compared to the remainder of the body, which is referred to throughout this disclosure as the “rear wall.” As a result, the front wall can be moved relative to the rear wall while conforming to the shape of the rear wall.

Because the oral airway front wall can be separated from the back wall, for example by pushing the front wall along the back wall until it passes from the end of the back wall, it is easier to take the airway out off the mouth following placement of an endotracheal tube without disturbing the endotracheal tube.

Modes for carrying out the present invention are explained below by reference to an embodiment of the present invention shown in the attached drawings. The above-mentioned object, other objects, characteristics and advantages of the present invention will become apparent from the detailed description of the embodiment of the invention presented below in conjunction with the attached drawings.

### BRIEF DESCRIPTION OF THE DRAWINGS

FIG. 1 is a perspective view of an oral airway.

FIG. 2 is perspective view of a portion of the oral airway of FIG. 1 in which the front wall is shown separated from the rear wall.

FIG. 3 is a schematic sectional view of the respective confronting surfaces of the front wall and rear wall illustrating the shape of the interlocking structures formed thereon.

FIG. 4 is a sectional view of the oral airway illustrating the regions of differing materials.

FIG. 5 is a side view of the oral airway of FIG. 1 showing the front wall in a retracted position relative to the rear wall.

FIG. 6 is a side view of the oral airway of FIG. 1 showing the front wall in an advanced position relative to the rear wall.

FIG. 7 schematically illustrates the oral airway of FIG. 1 positioned within the mouth of a patient, the oral airway configured with the front wall in a retracted position relative to the rear wall.

FIG. 8 schematically illustrates the oral airway of FIG. 1 positioned within the mouth of a patient, the oral airway configured with the front wall in an advanced position relative to the rear wall.

### DETAILED DESCRIPTION

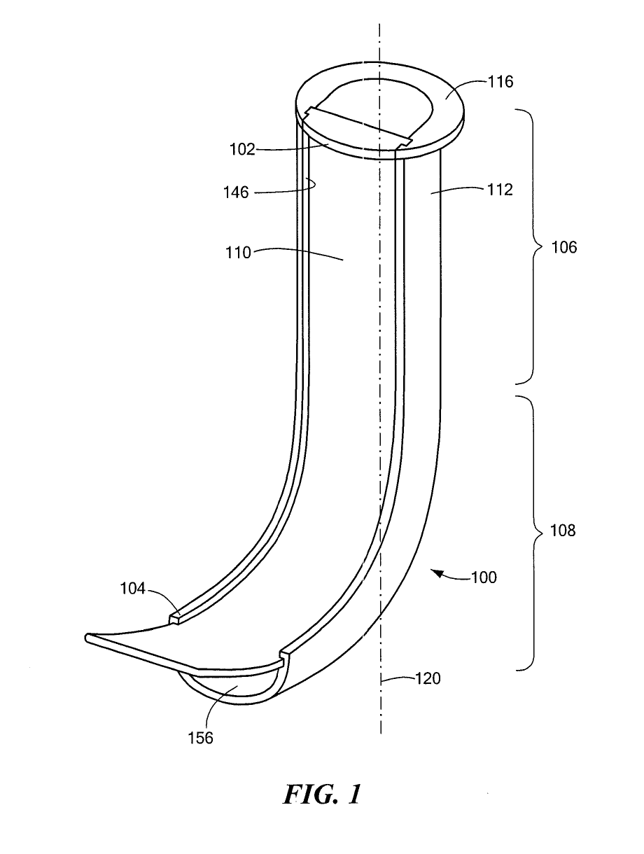

Referring now to FIG. 1, the oral airway device 100 is a tubular structure that includes a proximal end 102, and a distal end 104 that is opposed to the proximal end 102. The device 100 is formed having a linear portion 106 that includes the proximal end 102, and a curved portion 108 that extends from an end of the neck portion 106 and includes the distal end 104. A longitudinal axis 120 extends between the proximal end 102 and the distal end 104 so as to be concentric with a centerline of the linear portion 106. An outwardly protruding flange 116, provided about the circumference of the proximal end 102, serves to limit the extent to which the oral airway device 100 can be inserted into the oral cavity of a patient. The airway device 100 has a cross sectional shape that is generally oval, and that is uniform in dimension from the proximal end 102 to the distal end 104.

The airway device 100 includes a front wall 110, and a rear wall 112 that corresponding to the remainder of the airway device 100. The rear wall 112 is generally U-shaped in cross section, and the front wall 110 is secured to the rear wall 112 in such a way (discussed further below) as to bridge the open side 146 of the U-section, whereby the airway device 100 has a closed cross sectional shape.

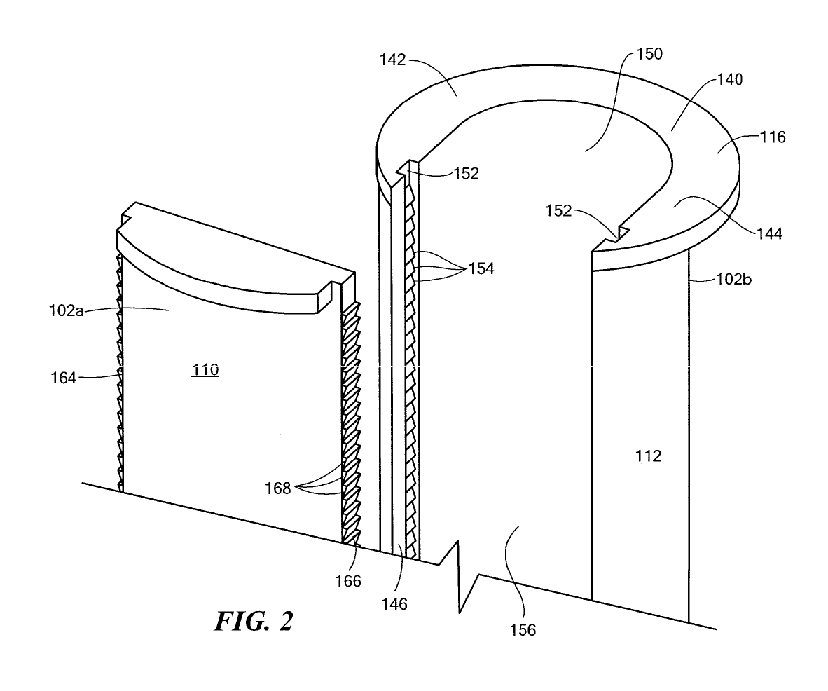

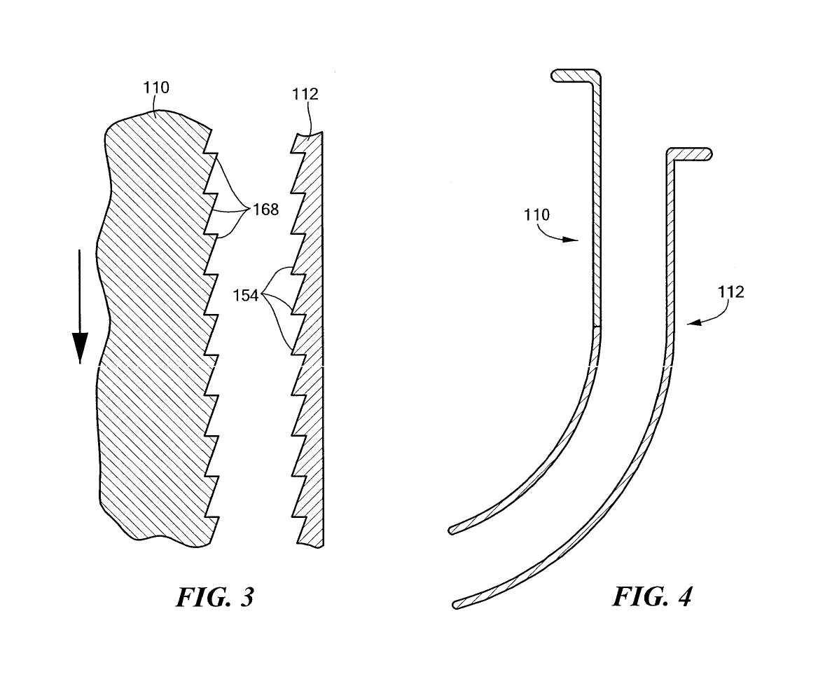

Referring also to FIGS. 2 and 3, and with reference to the U-shaped cross section of the rear wall 112, the U-shape provides a base 140 and a pair of legs 142, 144 that extend generally normally from the base 140. The base 140 is located on an opposed side of the airway device 100 from the front wall 110. The front wall 110 and the rear wall 112 each include portions corresponding to the linear portion 106 and to the curved portion 108. In particular, in the region corresponding to the curved portion 108, the front wall 110 is generally concave in shape, while the base 140 of the rear wall 112 is generally convex in shape.

Each leg 142, 144 is formed having a groove 152 located on an inner surface 150 of the rear wall 112. The grooves 152 are positioned adjacent to the open side 146 of the U-section such that the grooves 152 are mutually opposed and open facing each other across the open interior space 156 defined by the base 140 and legs 142, 144. Each groove 152 extends from the proximal end 102 to the distal end 104.

The front wall 110 is in the form of an elongated plate that is shaped and dimensioned so that lateral edges 164, 166 of the front wall 110 are received within the grooves 152 of the rear wall 112. As a result, the front wall 110 provides closure to the open side 146 of rear wall 112. In addition, the front wall 110 and rear wall 112 are maintained in an assembled configuration by interlocking structures formed on respective confronting surfaces of the rear wall grooves 152 and front wall lateral edges 164, 168. In the illustrated embodiment, the interlocking structures includes a series of spaced, wedge-shaped protrusions 154 formed on an interior surface of each groove 152, and at least one mating wedge-shaped protrusion 168 formed on each lateral edge 164, 166 of the front wall 110. The front wall mating protrusions 168 are configured to engage corresponding ones of the spaced protrusions 154 within the grooves 152. The respective engaged configuration can be overcome by applying an axial force to the front wall 110 sufficient to cause front wall mating protrusions 168 to slide over the groove protrusions 154. In addition, the protrusions 154, 168 are formed having the wedge-shape to limit relative movement within the groove between the front wall 110 and the rear wall 112 to one direction. In particular, the front wall can slide within the groove 152 relative to the rear wall 112 moving in a direction from the proximal end 102 to the distal end, but is prevented from moving in the opposite direction. Moreover, the series of spaced protrusions 154, 168 provide a series of stops, whereby the front wall 110 can be moved to a desired position relative to the rear wall 112, and then is retained in the desired position due to the interlocking engagement of the front and rear wall protrusions 154, 168.

Thus, the front wall 110 is configured to translate relative to the rear wall 112, while remaining engaged with the rear wall 112, and the translation is made in a direction generally parallel to the longitudinal axis 120. Moreover, the front wall 110 is retained in a desired position relative to the rear wall due to the interlocking protrusions 154, 168 formed on respective mating surfaces 152, 166. If desired, the front wall 110 can be completely separated from the rear wall 112 by forcing the front wall 110 along the grooves 152 to an extent that it exits the grooves 152.

Referring to FIG. 4, in this embodiment, the rear wall 112 is formed of a material that is sufficiently rigid to maintain the overall shape of the oral airway 100 while in use during an intubation procedure, and to provide a stable U-shaped cross sectional configuration. Since the oral airway 100 has a linear portion 106 and a curved portion 108 extending from one end of the linear portion 106, as the front wall 110 is moved within the grooves 152 relative to the rear wall 112, it must be able to transition between a linear configuration and a curved configuration. For this reason, at least a portion of the front wall 110 is formed of a second material that is flexible relative to the material (e.g, first material) used to form the rear wall 112. In some embodiments, the front wall 110 is formed so that at least the linear portion 106a of the front wall 110 is formed of the second material and a portion of the curved portion 108a of the front wall 110 is formed of the first material. In other embodiments, both the linear portion 106a and the curved portion 108a of the front wall 110 are formed of the second material. In still other embodiments, the front wall 110 is formed so that at least the linear portion 106a of the front wall 110 is formed of the second material and a portion of the curved portion 108a of the front wall 110 is formed of a third material that is different from both the first and second materials.

It is understood that the material used to form at least the linear portion 106a of the front wall 110 is sufficiently flexible to permit the portions of the front wall 110 disposed in the linear portion 106 to conform to the linear configuration of the linear portion 106, the portions of the front wall 110 disposed in the curved portion 108 to conform to the curved configuration of the curved portion 108, and to permit the front wall 110 to transition between the linear portion 106 and the curved portion 108. In addition, the material used to form the curved portion 108a of the front wall 110 is sufficiently stiff to permit the distal end 104 of the front wall 110 to lift the tongue when the oral airway is in use, as described below.

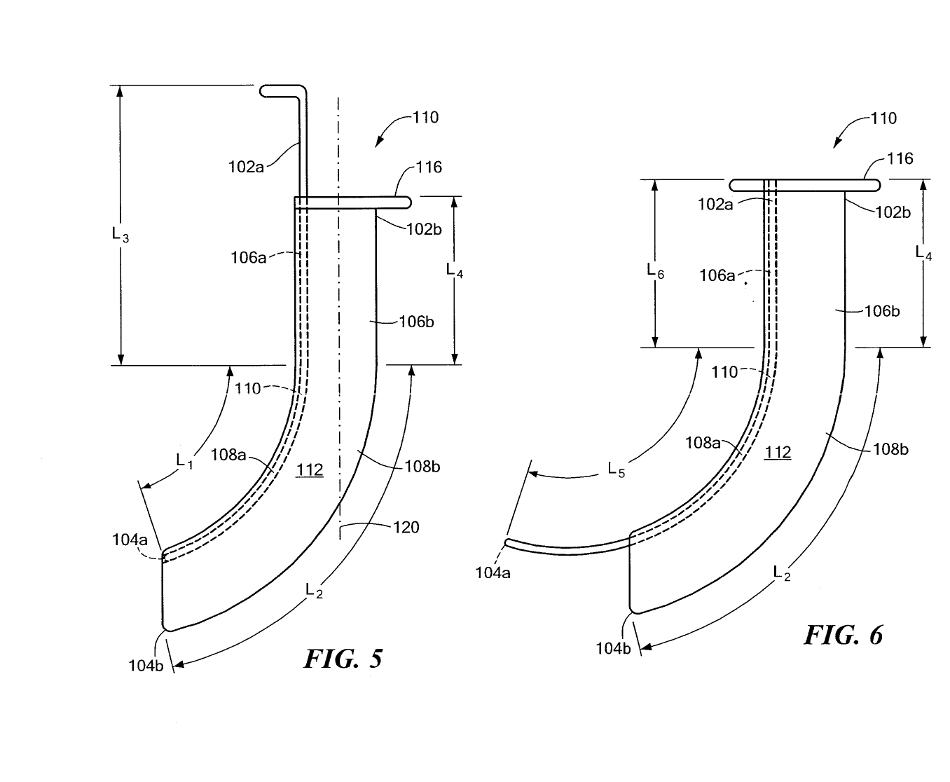

Referring to FIGS. 5 and 6, when the oral airway 100 is configured with the front wall 110 in a retracted position (FIG. 5), the curved portion 108a of the front wall 110 has a length L1 that is less than the length L2 of curved portion 108b of the rear wall 112, where length is defined as the distance along the device 100, for example in a direction from the proximal end 102 to the distal end 104. However, the linear portion 106a of the front wall 110 has a length L3 that is greater than the length L4 of the linear portion 106b of the rear wall 112. As a result, the proximal end 102a of the front wall 110 extends beyond the proximal end 102b of the rear wall 112, while the distal end 104a of the front wall 110 lies flush with the distal end 104b of the rear wall 112. This configuration is well suited for insertion of the oral airway 100 into the oral cavity of a patient.

When the oral airway 100 is configured with the front wall 110 in an advanced position (FIG. 6), the curved portion 108a of the front wall 110 has a length L5 that is greater than the length L2 of curved portion 108b of the rear wall 112. In addition, the linear portion 106a of the front wall 110 has a length L6 that is less than or equal to the length L4 of the linear portion 106b of the rear wall 112. As a result, the proximal end 102a of the front wall 110 lies flush with the proximal end 102b of the rear wall 112, while the distal end 104a of the front wall 110 extends beyond the distal end 104b of the rear wall 112. This configuration is well suited for pushing against the base of the tongue following insertion of the oral airway 100 into the oral cavity of a patient, as described below.

Thus, the respective lengths of the curved portion 108a and linear portion 106a of the front wall 110 vary depending on the relative position of the front wall 110 with respect to the rear wall 112.

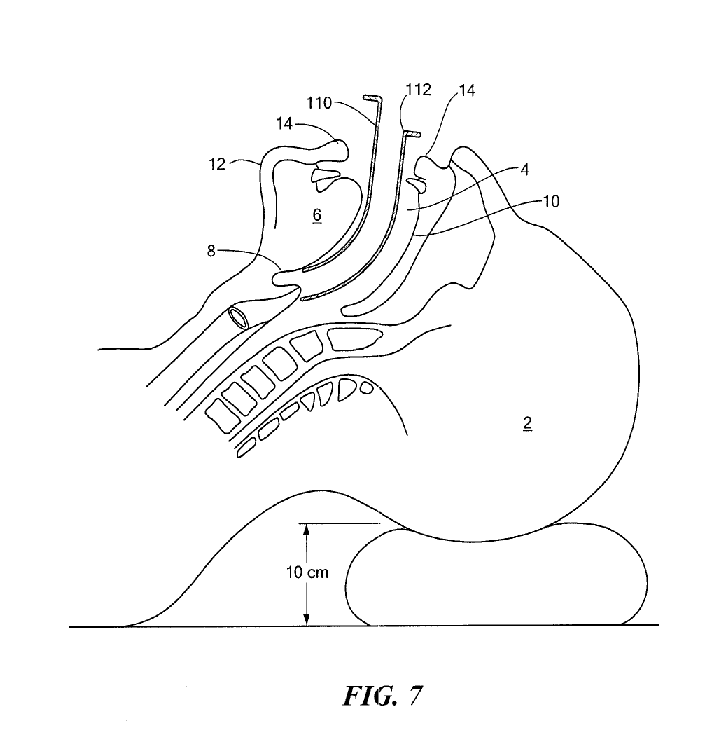

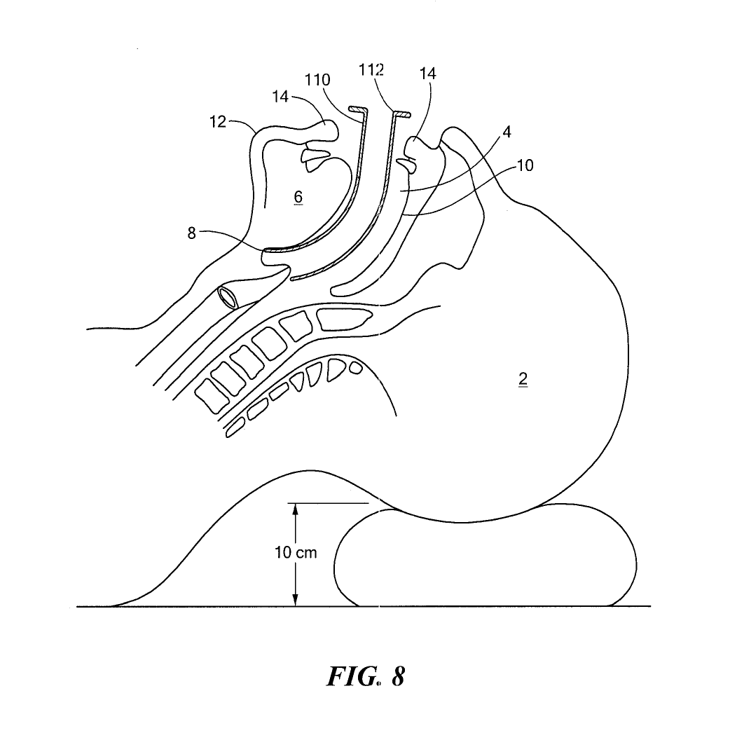

Referring to FIGS. 7 and 8, a method of using the oral airway to provide an unobstructed passageway for facilitating endotracheal intubation will be described.

In the first step, and prior to insertion of the oral airway 100 into the oral cavity 4 of a patient 2, the oral airway 100 is configured so that the front wall 110 is in the retracted position. As a result, the distal end 104a of the front wall 110 lies flush with the distal end 104b of the rear wall 112, providing a smooth, continuous leading edge that eases insertion and prevents patient trauma during insertion of the distal end 104 of the oral airway into the oral cavity 4.

In the second step, the oral airway 100 is inserted into the oral cavity 4 with the distal end 104 as the leading end. In addition, the oral airway 100 is oriented so that the front wall 110 faces the tongue 6, and so that the rear wall faces the palate 10. The oral airway 100 is inserted into the oral cavity until the distal end 104 is in the vicinity of the base of the tongue 8 and the proximal end resides in the mouth opening such that the flange 116 rests on the patient's lips (FIG. 7).

In the third step, the proximal end 102a of the front wall 110 is manually grasped and urged toward the distal end 104 of the oral airway 100. By application of sufficient force to overcome resistance to motion due to the engagement of the respective front and rear wall protrusions 154, 168, the front wall 110 is translated axially relative to the rear wall 112 so that the front wall 110 is moved toward the distal end 104. As a result, the front wall 110 is moved from the retracted position to the advanced position, and the distal end 104a of the front wall 110 extends beyond the distal end 104b of the rear wall 112b. Due to the curved configuration of the curved portion, the front wall distal end 104a is caused to contact the base of the tongue 8 and urge the base of the tongue toward the chin 12, exposing the vocal cords and providing an unobstructed passageway for facilitating endotracheal intubation (FIG. 8).

Although the oral airway 100 is illustrated as having a generally oval cross sectional shape, the device is not limited to this shape. For example, the oral airway may be formed having a circular or rectangular cross sectional shape. In addition, although the oral airway 100 is illustrated as having a closed sectional shape, the device is not limited to this configuration. For example, the oral airway may be formed having a U-shaped cross section that, when in place in the oral cavity, opens laterally (e.g., opens toward an ear of the patient). In this example, the rear wall comprises one leg and the base of the U-shape, and the front wall comprises the other leg of the U-shape.

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art. Moreover, while a working example of the present invention has been described above, the present invention is not limited to the working example described above, but various design alterations may be carried out without departing from the present invention as set forth in the claims.

What is claimed is,:

1. An oral airway comprising
a curved body including
a front wall which includes a concave portion of the body, and
a rear wall which includes a convex portion of the body,
wherein
the front wall is configured to translate relative to the rear wall, while remaining engaged with the rear wall, and
the rear wall is formed of a first material, and at least a portion of the front wall is formed of a second material, the second material being relatively flexible as compared to the first material.

2. The oral airway of claim 1, wherein the body further includes a proximal end, and a distal end opposed to the proximal end, and
the front wall translates relative to the rear wall in a direction extending from the proximal end to the distal end.

3. The oral airway of claim 1, wherein the front wall is separable from the rear wall.

4. The oral airway of claim 1, wherein the body further includes a linear portion and a curved portion extending from an end of the linear portion, and at least a portion the front wall linear portion is formed of the second material.

5. The oral airway of claim 4, wherein the front wall curved portion is formed of the first material.

6. The oral airway of claim 4, wherein the front wall proximal end is formed of the second material.

7. The oral airway of claim 1, wherein the front wall and rear wall are maintained in an assembled configuration by an interlocking structure.

8. The oral airway of claim 7, wherein the interlocking structure comprises a series of spaced protrusions formed on a side edge of the rear wall, and mating protrusions formed on a corresponding side edge of the front wall, wherein the mating protrusions are configured to engage corresponding ones of the rear wall spaced protrusions.

9. The oral airway of claim 1, wherein the body is tubular.

10. The oral airway of claim 1, wherein the front wall corresponds to about one-third of the circumference of the oral airway, and the rear wall corresponds to about two-thirds of the circumference of the oral airway.

11. The oral airway of claim 1, wherein an end of the linear portion includes an outwardly-protruding flange.

12. The oral airway of claim 1, wherein the body further includes a proximal end, and a distal end opposed to the proximal end, a linear portion including the proximal end, and a curved portion extending between the linear portion and the distal end, and
the front wall is selectively movable between a retracted position and an advanced position, such that
when in the retracted position, the front wall linear portion length is greater than the rear wall linear portion length, and the front wall curved portion length is less than the rear wall curved portion length, and
when in the advanced position, the front wall linear portion length is substantially equal to the rear wall linear portion length, and the front wall curved portion length is greater than the rear wall curved portion length,
where the wall length is the distance along the body between the proximal and distal ends.

13. The oral airway of claim 1, wherein the oral airway is generally oval in cross-sectional shape.

14. An oral airway comprising
a curved body including
a front wall which includes a concave portion of the body,
a rear wall which includes a convex portion of the body,
a proximal end,
a distal end opposed to the proximal end,
a linear portion including the proximal end, and
a curved portion extending between the linear portion and the distal end,
wherein
the front wall is configured to selectively translate relative to the rear wall, while remaining engaged with the rear wall, between a retracted position and an advanced position, such that
when in the retracted position, the front wall linear portion length is greater than the rear wall linear portion length, and the front wall curved portion length is less than the rear wall curved portion length, and
when in the advanced position, the front wall linear portion length is substantially equal to the rear wall linear portion length, and the front wall curved portion length is greater than the rear wall curved portion length.

15. The oral airway of claim 14, wherein the front wall translates relative to the rear wall in a direction extending from the proximal end to the distal end.

16. The oral airway of claim 14, wherein the front wall is separable from the rear wall.

17. The oral airway of claim 14, wherein the front wall and rear wall are maintained in an assembled configuration by an interlocking structure.

18. The oral airway of claim 17, wherein the interlocking structure comprises a series of spaced protrusions formed on a side edge of the rear wall, and mating protrusions formed on a corresponding side edge of the front wall, wherein the mating protrusions are configured to engage corresponding ones of the rear wall spaced protrusions.

19. The oral airway of claim 14, wherein the body is tubular.

20. The oral airway of claim 14, wherein the rear wall is formed of a first material, and at least a portion of the front wall is formed of a second material, the second material being relatively flexible as compared to the first material.

21. The oral airway of claim 20, wherein at least a portion the front wall linear portion is formed of the second material.

22. The oral airway of claim 20, wherein the front wall curved portion is formed of the first material.

23. The oral airway of claim 20, wherein the front wall proximal end is formed of the second material.

24. The oral airway of claim 20, wherein the front wall distal end is formed of the first material.

25. The oral airway of claim 14 wherein the oral airway is generally oval in cross-sectional shape.
